(19) United States
(12) Plant Patent Application Publication
(10) Pub. No.: US 2013/0055477 P1
(43) Pub. Date: Feb. 28, 2013

(54) Calycanthus plant named 'Aphrodite"

(50) ;

(71) Applicant: Thomas G. Ranney, Arden, NC (US)

(72) Inventor: Thomas G. Ranney, Arden, NC (US)

(21) Appl. No.: 13/317,289

(22) Filed: Oct. 13, 2011

(30) Foreign Application Priority Data
Aug. 25, 2011 (CA) 11-7360

(51) Int. Cl. A01H 5/00

(52) U.S. Cl. PLT/226

(57) ABSTRACT

Calycanthus ‘Aphrodite’ is a medium-sized, multi-stemmed shrub with glossy foliage. The large, purple-red, magnolia-like flowers have yellow tips on the inner tepals and a light fruity fragrance. The flowers are produced on long pedicles/peduncles at the distal end of the current year's growth for 6-8 weeks in late spring and early summer.

This application claims the benefit of Canadian Serial No. 11-7360 filed 25 Aug. 2011.

### GENUS AND SPECIES

Calycanthus chinensis×Calycanthus occidentalis

### VARIETY DENOMINATION

‘Aphrodite’

### BACKGROUND OF THE INVENTION

Calycanthus are deciduous flowering shrubs of the genus Calycanthaceae. Calycanthus floridus (also commonly referred to as the sweetshrub) is perhaps the most well known member of the genus, and is sometimes grown as an ornamental landscape plant, particularly in parts of the Eastern United States. Plant height can vary and ranges from 1 to 2.5 meters, growing as multi-stemmed shrubs. The flowers range in size (2-3 cm across) with tepals that vary from reddish purple to a dark chocolate brown. The fragrance is variable, but can have a pleasant fruity aroma.

Applicants believe there are additional opportunities for new and distinct Calycanthus as a landscape plant, e.g., for borders, foundations or naturalizing.

### SUMMARY OF THE INVENTION

The present invention comprises a new and distinct variety of Calycanthus, botanically known as Calycanthus L. ‘Aphrodite’, and hereinafter referred to by the varietal name ‘Aphrodite’. This new Calycanthus was developed through a breeding program at North Carolina State University, Mills River, N.C. ‘Aphrodite’ is an F₁ hybrid between Calycanthus chinensis and Calycanthus occidentalis. The first asexual propagation of ‘Aphrodite’ was carried out in June 2005 by rooting stem cuttings at the North Carolina State University, Mountain Horticultural Crops Research Station, Mills River, N.C. and has been asexually reproduced repeatedly by vegetative cuttings in North Carolina over a six year period.

‘Aphrodite’ roots readily from firm, terminal and subterminal, softwood cuttings treated with a basal dip of 5000 to 10,000 ppm indole butyric acid (potassium salt) in water. ‘Aphrodite’ has been found to retain its distinctive characteristics through successive asexual propagations.

‘Aphrodite’, when grown under normal horticultural practices at North Carolina State University, Mountain Horticultural Crops Research Station, Mills River, N.C., exhibited a number of distinguishing characteristics, including at least one of the following:

1. Large, purple-red, magnolia-like flowers with yellow apical margins on inner tepals.

2. Terminal and subterminal (axillary) flowers blooming over an eight week period providing a long bloom season.

3. Increased floral visibility due to flowers being held on stalks (pedicels and peduncles) above the foliage.

4. Lightly fragrant flowers with a fruity aroma.

5. Attractive glossy foliage.

### BRIEF DESCRIPTION OF THE PHOTOGRAPHS

This new Calycanthus plant is illustrated by the accompanying photographs which show the plant's form, foliage and flowers. The colors shown are as true as can be reasonably obtained by conventional photographic procedures. The photographs were taken in June 2011 of nine-year-old plants, growing under field conditions in Mills River, N.C.

FIG. 1 shows a typical flower.

FIG. 2 shows the sub-terminal, axillary flowers.

FIG. 3 shows the entire plant.

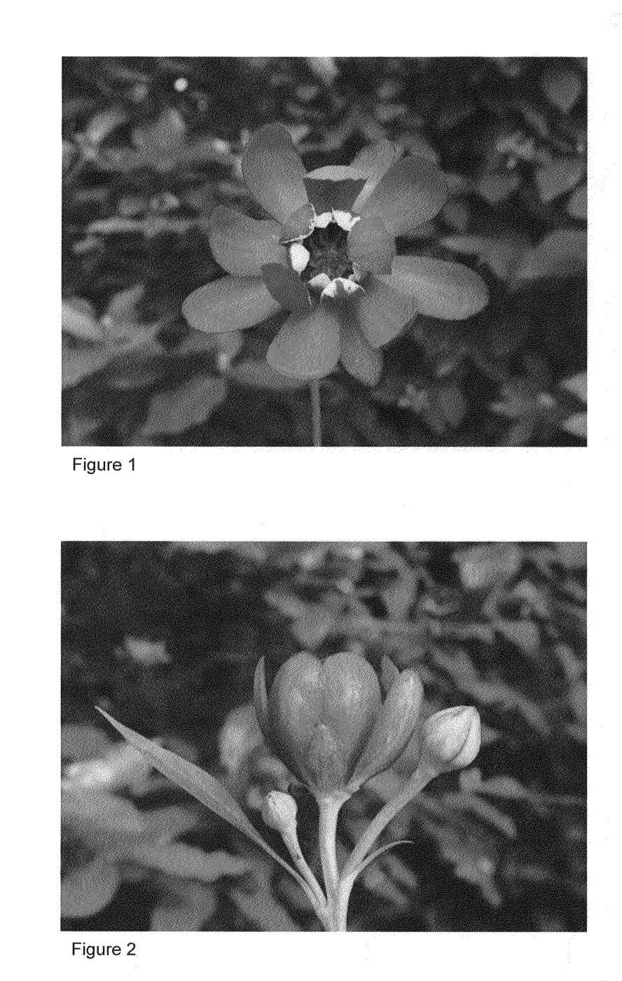

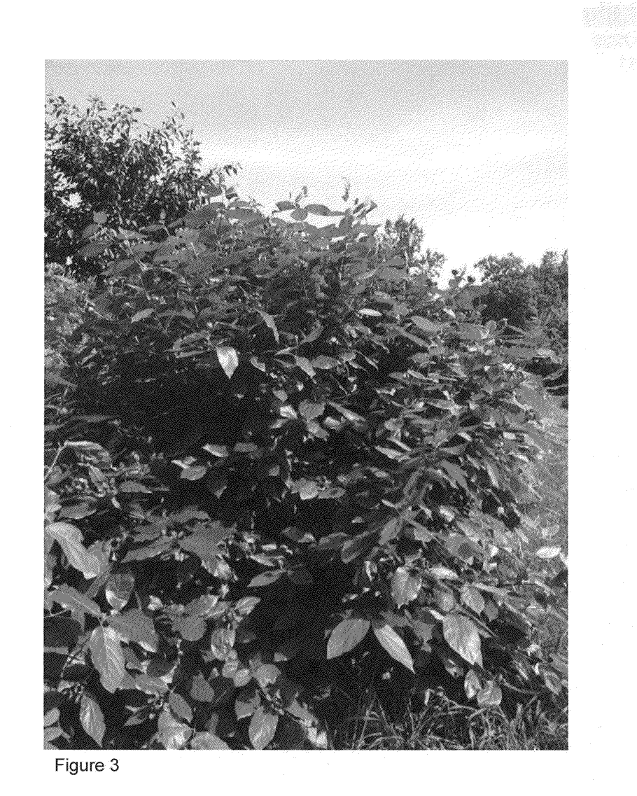

### DETAILED BOTANICAL DESCRIPTION

The following detailed description sets forth the distinctive characteristics of ‘Aphrodite’. The detailed description was taken on nine-year-old plants growing under field conditions in Mills River, N.C. in April 2008. Color references are to the R.H.S. Colour Chart of The Royal Horticultural Society of London (R.H.S.), 2001 Edition.
Classification: Shrub
Botanical name: Calycanthus L. ‘Aphrodite’
Common name: Sweetshrub
Parentage: Calycanthus chinensis×Calycanthus occidentalis
Plant description:
Growth habit: Multistemmed shrub
Plant shape: Globose
Height at maturity: 3-4 meters
Width at maturity: 3-4 meters
Vigor: Vigorous, shoots growing up to 0.5 meter in a season.
Stem:
   Immature stem texture.—Glabrous to tomentulose.
   Immature stem color.—RHS 144A (Yellow-Green).
   Mature stem texture.—Rugulose, glaborous.
   Mature stem color.—closest to RHS 199A (Greyed-Brown).
Trunk: typical diameter of nine-year-old plant 2 cm above the soil: 18.6 to 36.1 mm.
Branches:
   Length.—15 to 200 cm.
   Diameter.—5.5 to 13.6 mm.
Foliage:
   Type.—Deciduous.
   Arrangement.—Opposite.
   Shape.—Elliptical.
   Apex.—Acuminate to obtuse.
   Veins.—Type: Pinnate Adaxial & abaxial vein color: Closest to RHS 148C (Yellow-Green) Surface texture: Glabrous to tomentulose.
   Base.—Rounded to attenuate.
   Margin.—Entire.
   Mature leaf size.—Length: Average is 14.2 cm (range is 12.0 to 17.0 cm) Width at widest: Average is 6.8 cm (range is 5.3 to 7.5 cm).
   Immature leaf color.—Adaxial (upper) surface: Glossy, RHS 144A (Yellow-Green) Abaxial (lower) surface: Glaucesant Closest to RHS 144A (Yellow-Green).
   Immature leaf texture.—Adaxial surface: Scabridulous Abaxial surface: Interveinal areas glabrous with veins tomentulose.
   Mature leaf color.—Adaxial (upper) surface: Glossy, Closest to RHS 137A (Green) Abaxial (lower) surface: Glaucesant, RHS 137B (Green) Mature Leaf Texture: Adaxial: Scabridulous Abaxial: Interveinal areas glabrous, veins tomentulose.
   Fall foliage color.—yellow (20A) to golden brown (22A).
   Leaf attachment.—Petiolate.
   Petiole.—Shape: Laterally compressed cylinder with an adaxial groove. Length: Average is 10.8 mm (range is 7.86 to 14.96 mm) Width: Average is 2.2 mm (range is 1.71 to 2.61 mm).
   Surface.—Tomentulose to glabrous.
Inflorescence: Calycanthus ‘Aphrodite’ produces complete flowers on shoot terminals and frequently on subtending axils. Flowers have 26 to 30 tepals in a whorled series. Flowers are held on 16.6 to 26.5 mm stalks (pedicels and peduncles) at the distal end of the current season's growth. These flowers are magnolia-like and open with the greyed-orange apical margin of the inner tepals displayed prominently.
Flowering season: in Mills River, N.C. flowering begins in early May and continues through June and sporadically in July.
Lastingness of flower on the plant: 2 to 4 days
Fragrance: light fruity or melon-like fragrance
Self cleaning or persistent: Self Cleaning
Bud:
   Bud shape.—Orbicular.
   Bud diameter at balloon stage.—Average 19.61 mm (range is 15.42 to 21.89 mm).
   Bud length at balloon stage.—Average 22.65 mm (range is 15.42 to 26.55 mm).
   Bud color.—Margins of tepals RHS N186C (Greyed-Purple) with interior color of tepals and dominate color of bud RHS 183B to 183C (Greyed-Purple).
Perianth:
   Diameter at antithesis.—Average 84.83 mm (range is 78.43 to 92.48 mm).
Tepal:
   Number of tepals per flower.—Average 27 (range is 26 to 30).
   Outer tepal color, adaxial (upper) surface at anthesis.—Tepal margin RHS 59A (Red-Purple) with interior flushed with RHS 186A (Greyed-Purple).
   Outer tepal color, abaxial (lower) surfaces at anthesis.—RHS 59A (Red-Purple).
   Inner tepal color, adaxial surface.—RHS 59A (Red-Purple) with apical margin of tepal RHS 163D (Greyed-Orange).
   Inner tepal color, abaxial surface.—RHS 59A (Red-Purple).
   Surface texture and appearance.—Puberulous: fine hairs at right angles to the tepal surface, although tepal has texture and appearance of filamentous silk cloth.
   Shape.—Obovate.
   Apex.—Obtuse.
   Base.—Cuneate.
   Margin.—Entire.
   Outer tepal.—Length: Average is 38.84 mm (range is 36.77 to 40.33 mm) Width: Average is 17.19 mm (range is 16.49 to 18.14 mm).
   Inner tepal.—Length: Average is 18.45 mm (range is 17.85 to 19.29 mm) Width: Average is 8.94 mm (range is 8.55 to 9.62 mm).
   Tepals, fused or unfused.—Unfused.
Peducle/peduncle:
   Color.—RHS 144A (Yellow-Green).
   Length.—Average 20.89 mm (range is 16.58 to 26.48 mm).
   Width.—Average 3.25 mm (range is 2.37 to 3.73 mm).
   Shape.—Cylindrical.
   Surface texture.—Tomentulose.
Bract:
   Number.—3.
   Length.—Average 8.21 mm (range is 6.95 to 9.52 mm).
   Width.—Average 2.62 mm (range is 2.08 to 3.11 mm).
   Color.—RHS N144A (Yellow-Green).
   Shape.—Lanceolate.
   Apex.—Acuminate.
   Base.—Truncate.
Reproductive organs:
   Stamen.—Quantity per flower: Average 21 (range is 19 to 23) Shape: Linear to lanceolate. Anthers are longitudinally adnate to stalkly filament. Occassional petaloid stamens occur. Color at anthesis: RHS 10A (Yellow) and RHS 183A (Greyed-Purple) maturing to RHS 10A (Yellow) spotting with RHS 183A (Greyed-Purple) and RHS 10B (Yellow) Filament Length: Average is 1.9 mm (range is 1.3 to 2.2 mm) Width of filament: Average 1.0 mm (range is 0.08 to 1.4 mm).
   Anther.—Length: Average is 5.63 mm (range is 5.00 mm to 7.00 mm) Width at widest: Average is 1.2 mm (range is 1.1 to 1.5 mm) Dehiscence: Longitudinally Anther Color: closest to RHS 186A (Greyed-Purple) Pollen color: RHS 10B (Yellow) Attachment: Longitudinally adnate to filament.
   Pistil.—Style Length: Average 7.25 mm (range 7.00 to 8.00 mm) Style Width at midpoint: Filamentitious less than 0.5 mm Ovary diameter: Average 13.6 mm (range 12.00 to 19.00 mm).
   Fruit/seed set.—Fruit: Not currently available.
Disease and insect resistance: Cultural requirements, disease and pest resistance/susceptibility for Calycanthus ‘Aphrodite’ appear to be similar to other Calycanthus. This variety can be grown in full sun to shade with best growth on moist sites that do not become overly dry. Calycanthus ‘Aphrodite’ tolerates a range of soil pH from about 4.5 to 6.5.
Comparison with parental lines and known cultivars: ‘Aphrodite’ has primarily purple-red tepals, an imbricate tepal arrangement, and a light fruity fragrance while the female parent, C. chinensis, has a two-series tepal arrangement with the whitish-pink outer tepals and yellow inner tepals and no fragrance. ‘Aphrodite’ has large flowers, 78-93 mm in diameter with inner tepals having a yellow distal margin while the male parent, C. occidentalis, has smaller flowers, 20-40 mm in diameter, with reddish-brown tepals.

Table 1 shows the differences between ‘Aphrodite’, Calycanthus ‘Venus’ U.S. Plant Pat. No. 15,925 and Calycanthus ‘Hartlage Wine’ (unpatented).

TABLE 1
COMPARISON TO COMMERCIAL CULTIVARS

| | Cultivar | | |
|---|---|---|---|
| Trait | ‘Aphrodite’ | ‘Venus’ | ‘Hartlage Wine’ |
| Diameter | 7.8 to 9.3 cm | 7.5 to 11.5 cm | 6.9 to 8.5 cm |
| of Flower | | | |
| Color | Outer Tepals: | Outer Tepals: | Outer Tepals: Margins |
| of Flower | margin RHS 59A | RHS 155A | RHS 187B |
| | (Red-Purple) | (White) | (Greyed-Purple) with |
| | with interior | Inner Tepals: | interior RHS 53A |
| | flushed with RHS | RHS 155A | (Red) |
| | 186A | (White) with | Inner Tepals: RHS |
| | (Greyed-Purple) | apical margin | 187B (Greyed-Purple) |
| | Inner Tepals: | RHS 5C | with apical margins |
| | RHS 59A | (yellow) with | RHS 163C (Yellow) |
| | (Red-Purple) | blush of RHS | |
| | with apical | 60C | |
| | margin of tepals | (Red-Purple) | |
| | RHS 163D | on edge and | |
| | (Greyed-Orange) | base | |
| Flower | Light fruity | Fruity | No Fragrance |
| Fragrance | | | |
| Tepal | Open flower with | Outer tepals | Outer tepals cup to |
| Presentation | yellow apical | opening fully to | partial obscure |
| | margin of inner | display the | creamy white to yellow |
| | tepals | yellow and red | apical margins of inner |
| | prominently | inner whorl on | tepals. |
| | displayed | some flowers | |
| | | while outer | |
| | | tepals partially | |
| | | obscures inner | |
| | | tepals on other | |
| | | flowers | |
| Bud Color | Margins of tepals | Outer tepals | Margins of tepals |
| | RHS N186C | are primarily | closest to RHS N186 |
| | (Greyed-Purple) | RHS 1D | (Greyed-Purple) with |
| | with interior color | (green-yellow) | interior color of tepals |
| | of tepals and | with some | and dominate color of |
| | dominate color of | ranging from | bud RHS 187B |
| | bud RHS 183B | RHS 144C to | (Greyed-Purple) |
| | to 183C | RHS 144D | |
| | (Greyed-Purple) | (Yellow-Green) | |

I claim:

1. A new and distinct variety of Calycanthus plant as shown and described herein.
